(19) United States
(12) Patent Application Publication
(10) Pub. No.: US 2026/0191797 A1
(43) Pub. Date: Jul. 9, 2026

(54) AMPHIPHILIC POLY(AMINO ACID) LINEAR BLOCK COPOLYMERS AND NANOPARTICLES THEREOF FOR DRUG DELIVERY APPLICATIONS

(71) Applicant: Nanothera Biosciences Inc., Mount Laurel, NJ (US)

(72) Inventors: Coralie LEBLEU, BREUILLET (FR); Laetitia PLET, NOISY-SUR-ECOLE (FR); Christiane PHILIPPE, D'HUISON LONGUEVILLE (FR); Gauthier ERRASTI, ORSAY (FR); Thomas DELACROIX, MENNECY (FR); Sébastien LECOMMANDOUX, CANEJAN (FR); Raj CHAKRABARTI, Moorestown, NJ (US)

(73) Assignee: Nanothera Biosciences Inc., Mount Laurel, NJ (US)

(21) Appl. No.: 18/868,491

(22) PCT Filed: May 23, 2023
(86) PCT No.: PCT/EP2023/063848

§ 371 Date: Nov. 22, 2024

Related U.S. Application Data
(60) Provisional application No. 63/344,807, filed on May 23, 2022.

(30) Foreign Application Priority Data
May 23, 2022 (EP) 22305766.2

(51) Int. Cl. A61K 9/51; C08L 77/04; C08L 79/02

(52) U.S. Cl. CPC A61K 9/5146; A61K 9/5192; C08L 77/04; C08L 79/02; C08L 2203/02

(57) ABSTRACT

The present invention relates to the field of polymer chemistry and more particularly to poly(amino acid) copolymers and uses thereof for drug delivery applications. In particular, the present invention concerns a linear copolymer comprising a polysarcosine block, pSar, containing from 15 to 99 sarcosine constitutional units and a poly(amino acid) block, pAA, containing from 8 to 120 amino acid constitutional units.

### FIELD OF THE INVENTION

The present invention relates to the field of polymer chemistry and more particularly to poly(amino acid) copolymers and uses thereof for drug delivery applications.

### BACKGROUND OF THE INVENTION

Active pharmaceutical ingredients (APIs) often need to be mixed with one or several excipients to form ready to use drug products. Excipients are inactive ingredients that usually have a specific function in the drug form: they can be used for example as binders, lubricants, coating agents, or filling agents. Depending on the therapeutic indication, drugs can be administered through different routes (e.g. topical, oral, parenteral) which require different final drug dosage forms and thus the use of suitable excipients.

Polymer excipients are widely used in the formulation of drug products intended for every possible route of administration. They include natural compounds such as cellulose, semi-synthetic compounds such as cellulose derivatives, and synthetic polymers like poly(ethylene glycol) (PEG), polylactides, or polyamides. Polymer excipients can be used to modify the physical or chemical properties of an API, for example change its viscosity or increase its solubility.

Amphiphilic block copolymers are particularly interesting excipients to solubilize hydrophobic APIs in water. Constituted at least of one hydrophilic block covalently bound to one hydrophobic block, they self-assemble in water to form nano-sized aggregates at or above their critical aggregate concentration (CAC). These resulting supramolecular assemblies, also called nanoparticles (NPs), usually have a size ranging from 10 to 100 nm and can have various morphologies. For example, a micelle has a spherical core-shell structure where the hydrophobic blocks formed the hydrophobic core that is stabilized by a hydrophilic shell constituted of the hydrophilic blocks. A hydrophobic API can be loaded and segregated in the core during the micellization. API-loaded micelles prepared in diluents commonly used for parenteral administration such as saline can increase the solubility of the APIs without the need for co-solvents such as Cremophor® EL (polyoxyethylated castor oil) that is known to cause several adverse effects. Another advantage is that the API is not covalently bound to the copolymer, this drug form could thus be applied to a broad range of APIs.

Furthermore, in addition to increasing the water-solubility of APIs, nanoparticles offer various advantages such as preventing the API premature degradation, controlling its release, improving its bioavailability, and for anti-tumor applications, enhancing its absorption into tumor tissues by passive targeting through the enhanced permeation and retention effect (EPR effect).

PEG is the most commonly used hydrophilic block due to its stealth effect. However, PEG is not biodegradable and can trigger immunogenic responses at high dosage or in long-term administration.

Polysarcosine (pSar) is a good alternative to PEG since they share similar properties such as hydrophilicity, stealthiness and low toxicity but pSar has the advantage of being biodegradable as it is based on the endogenous amino acid derivative sarcosine, N-methylated glycine.

Amphiphilic polysarcosine-poly(amino acid) copolymers have been recently developed for their biodegradability and ability to self-assemble in water into nanoparticles.

U.S. Ser. No. 10/836,869 discloses the use of such type of copolymers to solubilize hydrophobic molecules. These multiblock copolymers comprise a large number of sarcosine and amino acid units which lead to costly synthesis and post purification and industrial scale-up challenges.

Therefore, there is still a need for a polymer capable of solubilizing hydrophobic APIs that does not exhibit side effect when used for drug delivery applications.

### OBJECT OF THE INVENTION

The invention is directed to a linear copolymer comprising a polysarcosine block, pSar, containing from 15 to 99 sarcosine constitutional units and a poly(amino acid) block, pAA, containing from 8 to 120 amino acid constitutional units.

Preferably, the linear copolymer has a hydrophilic fraction, f(pSar), ranging from 5 to 80%, preferably from 10 and 70%, wherein f(pSar) is the ratio in percentage of number average molar mass of the pSar block on the number average molar mass of the copolymer.

Preferably, pAA block has a H hydrophobicity coefficient equal or higher than −0.50, preferably equal or higher than −0.25, preferably equal or higher than 0.00, preferably equal or higher than 0.50 and more preferably equal or higher than 0.80.

Preferably, amino acid constitutional units of pAA block are derived from hydrophobic amino acid which is preferably selected from the group comprising alanine, valine, norleucine, leucine, isoleucine, methionine, phenylalanine, tryptophan, proline, tyrosine, their derivatives, protected hydrophilic amino acids and combinations thereof.

Derivatives of hydrophobic amino acids are preferably protected hydrophobic aminoacids.

Preferably, the copolymer is selected from copolymer of formula I or II,

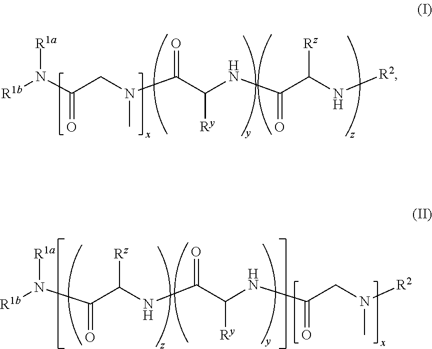

wherein
x is the number of sarcosine constitutional unit and is an integer ranging from 15 to 99,
y+z is the number of amino acid constitutional units and is an integer ranging from 8 to 120,
groups R^{y} and R^{z} are independently chosen from an amino acid lateral-chain group,
groups R^{1a} and R^{1b} are independently chosen from H, alkyl, alkenyl, alkynyl, cycloalkyl, aryl and heteroaryl,
group R² is chosen from H and a nitrogen protecting group.

Preferably, x is an integer ranging from 20 to 95, more preferably from 24 to 85.

Preferably, y+z is an integer ranging from 8 to 110, more preferably from 12 to 100.

Preferably, y+z is an integer ranging from 8 to 50, more preferably from 9 to 45, even more preferably from 12 to 40.

Preferably, y+z is an integer ranging from 55 to 110, more preferably from 60 to 100.

Preferably, y is a number ranging from 0 to 120, more preferably from 0 to 110, preferably from 5 to 100, preferably from 9 to 95, preferably from 10 to 90.

Preferably, z is a number ranging from 0 to 120, more preferably from 0 to 110, preferably from 5 to 100, preferably from 9 to 95, preferably from 10 to 90.

At least one of z or y is different from 0.

Preferably R^{y} is the lateral chain of a hydrophobic L-amino acid and R^{z} is the lateral chain of a hydrophobic D-amino acid.

Preferably R^{y} is chosen from the lateral chain of L-leucine, L-phenylalanine, L-tyrosine, γ-benzyl-L-glutamate, γ-tert-butyl-L-glutamate and L-cyclohexylglycine.

Preferably R^{z} is chosen from the lateral chain of D-leucine, D-phenylalanine, D-cyclohexylglycine, D-tyrosine, γ-benzyl-D-glutamate and γ-tert-butyl-D-glutamate.

The invention is also directed to a method of preparation of a linear copolymer described above by polymerization of sarcosine derivates and amino acid derivates, wherein derivates are represented by formulas III and IV as follows

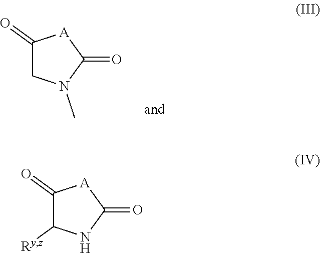

wherein
A is O or S,
R^{y}, R^{z} are as defined above.

The invention is also directed to nanoparticles comprising a linear copolymer as described above or prepared according to the method described above.

Preferably, nanoparticle further comprises at least one active compound, preferably selected from hydrophobic compounds, in particular hydrophobic active pharmaceutical ingredients.

Nanoparticle preferably satisfies at least one of the following conditions:
a hydrodynamic diameter lower than 400 nm, preferably ranging from 5 to 200 nm;
a polydispersity index lower than 0.70, preferably ranging from 0.02 to 0.70;
a loading efficiency of the active compound of more than 20%, preferably more than 30%.

The invention is further directed to a method of preparing nanoparticles as described above comprising the following steps:
preparing an organic solution containing the copolymer of the invention,
then, mixing the organic solution with an aqueous solution upon stirring,
then, removing the organic solvent.

Preferably nanoparticle further comprises an active compound and the method comprises the following steps:
preparing an organic solution containing the copolymer of the invention and at least an active compound,
then, mixing the organic solution with an aqueous solution upon stirring,
then, removing the organic solvent.

### Definitions

The following are definitions of various terms used herein to describe the present disclosure and are further illustrated by the embodiments, sub-embodiments, and compounds disclosed herein. These definitions apply to the terms as they are used throughout this specification unless otherwise indicated in specific instances, either individually or as part of a larger group.

The term “halogen” or “halo”, as used in the present invention, refers to a fluorine, chlorine, bromine or iodine atom.

The term “alkyl”, as used in the present invention, refers to a straight or branched monovalent saturated hydrocarbon chain. “(C₁-C₁₀) alkyl” refers to alkyl containing from 1 to 10 carbon atoms.

The term “alkenyl” is given its ordinary meaning in the art and refers to an unsaturated hydrocarbon chain. “(C₂-C₁₀) alkenyl” refers to alkenyl containing from 2 to 10 carbon atoms that includes one or more carbon-carbon double bonds.

The term “alkynyl” is given its ordinary meaning in the art and refers to an unsaturated hydrocarbon chain. “(C₂-C₁₀) alkynyl” refers to alkynyl containing from 2 to 10 carbon atoms that includes one or more carbon-carbon triple bonds.

The term “cycloalkyl”, as used in the present invention, refers to a hydrocarbon ring. “(C₃-C₁₀) cycloalkyl” refers to cycloalkyl having 3 to 10 carbon atoms including, but not limited to, cyclopropyl, cyclobutyl, cyclopentyl, cyclohexyl.

The term “aryl”, as used in the present invention, refers to an aromatic hydrocarbon group preferably comprising 6 to 10 carbon atoms, comprising one or more fused rings and eventually one or more substituents such as halogen, (C₁-C₆) alkyl, —O(C₁-C₆) alkyl. For example, aryl includes phenyl, benzyl or naphthyl group.

The term “heteroaryl”, as used in the present invention, refers to an aryl comprising one or several, notably one or two, preferably one, fused hydrocarbon cycles in which one or several, notably one to four, advantageously one or two, carbon atoms each have been replaced with a heteroatom selected from a sulfur atom, an oxygen atom and a nitrogen atom, preferably selected from an oxygen atom and a nitrogen atom, in particular a nitrogen atom. It can be a benzothiazolyl, furyl, thienyl, pyrrolyl, oxazolyl, isoxazolyl, thiazolyl, isothiazolyl, imidazolyl, pyrazolyl, oxadiazolyl, thiadiazolyl, triazolyl, tetrazolyl, pyridyl, pyridazinyl, pyrimidinyl, pyrazinyl, triazinyl, quinolyl, isoquinolyl, quinoxalyl or indolyl.

The terms “units” or “constitutional units” in the present disclosure refers to the constitutional units derived from polymerization of the monomer.

The term “linear copolymer” refers to copolymer containing pSar block and pAA block with a linear structure for example as illustrated in formula (I) or (II). The term “linear copolymer” does not encompass a nonlinear copolymer such as a multi-arm star shaped polymer and more particularly 3-arm star shaped copolymer.

The terms “sarcosine constitutional unit” or “sarcosine unit” refer to unit derived from the monomer of sarcosine.

The terms “amino acid constitutional unit” or “amino acid unit” refer to unit derived from the monomer of amino acid.

The term “AA” is used to designate an amino acid. In the present disclosure, the term “AA” does not encompass sarcosine.

Amino acids are represented by formula NH₂—CHR—CO₂H, wherein R represents the lateral chain group of each amino acid. Amino acids used in the present invention can be natural amino acids or unnatural amino acids. The term “natural amino acids” refers to any naturally occurring amino acid that can be found in proteins or in nature. These are L-alanine, L-arginine, L-asparagine, L-aspartate, L-cysteine, L-glutamate, L-glutamine, glycine, L-histidine, L-isoleucine, L-leucine, L-lysine, L-methionine, L-phenylalanine, L-proline, L-pyrrolysine, L-selenocysteine, L-serine, L-threonine, L-tryptophan, L-tyrosine, and L-valine.

The term “unnatural amino acid” refers to any amino acid that is not included in the list of naturally occurring amino acids. Unnatural amino acid includes amino acid wherein the lateral chain “R” is chemically modified for example by functionalization or protecting groups.

The term “amino acid lateral chain group” also defined as “lateral chain group” refers to the group R on the α-carbon of any natural or unnatural AA, as defined herein. For example, the lateral chain group of L-alanine is a methyl, and the lateral chain group of γ-benzyl-glutamate is group —(CH₂)₂COCH₂C₆H₅.

The term “D-amino acid” refers to an amino acid wherein the alpha carbon of the AA is in the D-configuration, also designated as D isomer, and the term “L-amino acid” refers to an amino acid wherein the alpha carbon of the AA is in the L-configuration, also designated as L isomer.

The term “poly(amino acid)” or “pAA” is used to designate a block made of a linear polymeric chain where the constitutional units are derived from amino acids or a mix of amino acids and are covalently linked by peptide bonds. A pAA can be formed via a ring-opening polymerization of monomers of amino acid. When only L-amino acid monomers are used, the resulting block is a poly(L-amino acid) block. When only D-amino acid monomers are used, the resulting block is a poly(D-amino acid) block. When a mix of L and D-amino acid is used in an unspecified sequence arrangement, the resulting block is a poly(L-amino acid-co-D-amino acid) block.

The term “Sar” is used to designate sarcosine.

The term “polysarcosine” or “pSar” is used to designate a block made of a linear polymeric chain where the constitutional units derive from sarcosine and are covalently linked by peptide bonds.

The term “monomer of amino acid” or “amino acid monomer” refers to an amino acid or any amino acid derivate which can be used in a polymerization process.

The term “monomer of sarcosine” refers to sarcosine or any sarcosine derivate which can be used in a polymerization process.

The term “sarcosine derivate” or “amino acid derivate” refers to any reactive form of sarcosine or amino acid which can be used in a polymerization process. For example, for a ring-opening polymerization, derivates can be a N-carboxyanhydride (NCA) of amino-acid or of sarcosine or a N-thiocarboxyanhydride (NTA) of amino-acid or of sarcosine.

The term “initiator” or “polymerization initiator” in the present disclosure refers to a molecule that reacts with a monomer to form a compound capable of reacting successively with other monomers by chain propagation to form a polymer.

The term “terminating agent” refers to a compound that reacts with the end of a living polymer chain and stops the chain propagation.

The term “capping agent” refers to a compound that reacts with the N-terminal part of the polymer chain to covalently bind a functional group such as —C(O)CH₃.

The terms “drug-loaded” and “loaded” refers to a particle comprising an active compound such as an API entrapped within the particle for example by encapsulation.

The expression “pharmaceutically acceptable” as used in the present invention is intended to mean what is useful to the preparation of a pharmaceutical composition, and what is generally safe and non-toxic, for a pharmaceutical use.

The terms “drug”, “Active Pharmaceutical Ingredient”, “API”, “pharmaceutical” and derivatives thereof, are used interchangeably and refer to a substance intended for use in the diagnosis, cure, mitigation, treatment, or prevention of a disease.

The terms “particle” and “nanoparticle” according to the present invention are used interchangeably to designate an object obtained by the spontaneous self-assembly of block copolymers. For example, these nanoparticles can be micelles, polymersomes or polyplexes.

By “polymeric micelle” it is meant an object characterized by a core-shell structure with a hydrophobic core and a hydrophilic shell, formed by self-assembly of amphiphilic copolymers.

By “polymersome” it is meant a polymer-based vesicle which is an object with a structure of a bilayer enclosing an aqueous compartment, formed by self-assembly of amphiphilic copolymers.

By “polyplex” it is meant a polymeric system containing a complexed nucleic acid (eg DNA or RNA), formed by electrostatic interactions between cationic groups of a polymer and negatively charged nucleic acid.

It is understood that any one of the embodiments and preferential or advantageous disclosures described below can be combined with any one of the other listed embodiments, preferential or advantageous disclosures.

### METHODS

NCA polymerization can be monitored by Fourier-transform infrared spectroscopy (FTIR). For example, polymerizations are considered completed when NCA-associated carbonyl bands at 1850 and 1778 cm⁻¹ disappeared which correspond to the total consumption of NCAs.

The number of constitutional units of each monomer of pSar block and of pAA block can be determined by proton (¹H) nuclear magnetic resonance (NMR).

The copolymer's number average molar mass (Mₙ) and dispersity (Ð) can be determined by size-exclusion chromatography (SEC).

Hydrodynamic diameter (Dₕ) and polydispersity index (PDI) of the particles can be determined by dynamic light scattering (DLS).

The loading efficiency (LE) can be determined using ultra performance liquid chromatography (UPLC).

The loading content (LC) can be determined using SEC.

The water solubility of the API can be determined by any experimental or analytical method, such as UPLC.

The H hydrophobicity coefficient of pAA block of the linear copolymer according to the invention can be determined as follows.

The hydrophobicity coefficient of a block depends on the length of the block, and/or on its composition. The hydrophobicity coefficient H is defined by the following equation E1.

$$H = A log P / n \qquad Equation E1$$

wherein,
   “H”: is the hydrophobicity coefficient of the pAA block;
   “Alog P”: is the simulated octanol-water partition coefficient of the PAA block with an amide function on its C-terminal end and an acetamide function on its N-terminal end and no matter if the amino acid of the PAA block has D-amino acid or L-amino acid configuration; and
   “n”: is the number of AA units in the pAA block (i.e. in particular n=y or n=z and one of y or z is equal to 0).

When the pAA block is composed of different AA units, i.e. in particular when y and z are both different from 0, the H coefficient is determined as the arithmetic mean of H coefficients for each type of AA units.

For example, with a block derived from 2 amino-acid monomers P(AA1y-co-AA2z), the H coefficient is defined by the equation E2 below.

$$HP = ( AA 1 y - co - AA 2 z ) = ( yHAA 1 + zHAA 2 ) / ( y + z ) \qquad Equation E2$$

Alog P is determined, for a pAA block, by the predictive model for the octanol-water partition coefficient defined by Ghose and Crippen and calculated with Biovia Draw 19.1.NET

### -----Version-----

MDL.Draw.Editor 19.1.0.1792
   -----Referenced and loaded assemblies-----
MDL.Cheshire: loaded version=5.1.0.22
MDL.CheshireLic: loaded version=5.1.0.22
MDL.Cslnline: referenced and loaded version=3.0.4.47
MDL.Draw.Clipboard: referenced and loaded version=19.1.0.1792
MDL.Draw.Editor: loaded version=19.1.0.1792
MDL.Draw.Foundation: referenced and loaded version=19.1.0.1792
MDL.Draw.Foundation. PPChemAPIIO: loaded version=19.1.0.1792
MDL.Draw.Renderer: referenced and loaded version=19.1.0.1792
MDL.Draw.TextServicesWrapper: referenced and loaded version=19.1.0.1792
nalpp: referenced version=19.1.0.1963
SandBar: referenced and loaded version=1.4.3.1
SandDock: referenced and loaded version=3.0.6.1.

The model of Ghose and Crippen refers to the model described in the following articles: Ghose, A. K.; Crippen, G. M. Atomic Physicochemical Parameters for Three-Dimensional-Structure-Directed Quantitative Structure-Activity Relationships. 2. Modeling Dispersive and Hydrophobic Interactions. J. Chem. Inf. Comput. Sci. 1987, 27 (1), 21-35. https://doi.org/10.1021/ci00053a005.

Ghose, A. K.; Viswanadhan, V. N.; Wendoloski, J. J. Prediction of Hydrophobic (Lipophilic) Properties of Small Organic Molecules Using Fragmental Methods: An Analysis of ALOGP and CLOGP Methods. J. Phys. Chem. A 1998, 102 (21), 3762-3772.

Specific protocols for each method are given in the examples.

### DETAILED DESCRIPTION OF THE INVENTION

Surprisingly, it has been found that linear copolymers having a restricted number of Sar units are able to increase the solubility of active compounds and preferably of hydrophobic compounds such as hydrophobic APIs.

Advantageously, linear copolymers according to the invention are less expensive than copolymers of the prior art because their synthesis is facilitated. Furthermore, industrial scale-up of the copolymers according to the invention can also be facilitated.

In particular, linear copolymers according to the invention, via their ability to self-assemble into nanoparticles, are capable of improving the water solubility of hydrophobic compounds such as hydrophobic APIs preferably with a factor greater than 100, preferably greater than 200, preferably greater than 500, preferably greater than 1000, preferably greater than 2000 and more preferably greater than 3000.

### COPOLYMER

The invention relates to a linear copolymer comprising:
a polysarcosine block, pSar, containing from 15 to 99 sarcosine constitutional units; and
a poly(amino acid) block, pAA, containing from 8 to 120 amino acid constitutional units.

The person skilled in the art will understand that when the pAA block is formed by different AAs, the order of each AA unit can be statistical or controlled.

One advantage of copolymers according to the invention is that they are stable, biocompatible and easily degraded in vivo.

According to the invention, the pSar block of the copolymer is hydrophilic whereas the pAA block is less hydrophilic than the pSar block and thus has a hydrophobic behavior. Those characteristics render the copolymer amphiphilic and allow it to form stable nanoparticles such as micelles. Furthermore, nanoparticles formed by copolymers of the invention are biocompatible and easily degraded in vivo.

The amphiphilic character of the pSar-pAA copolymers according to the invention can be used to form nanoparticles. Advantageously, said nanoparticle can encapsulate an active compound for example to limit its degradation or to increase its solubility in water.

The limited number of constitutional units of the pSar-pAA copolymers of the invention makes the preparation of each block easy and cost-attractive.

pSar block contains a chain of from 15 to 99 sarcosine units, preferably from 20 to 95, preferably from 24 to 85 and more preferably from 28 to 80.

In one preferred embodiment, pSar block contains a chain of from 50 to 99 sarcosine units, preferably from 55 to 95, preferably from 60 to 85 and more preferably from 65 to 80.

In another preferred embodiment, pSar block contains a chain of from 15 to 50 sarcosine units, preferably from 18 to 45, preferably from 22 to 40 and more preferably from 25 to 35.

The AA of which the pAA are composed are preferably selected in the lists disclosed further below. The selection is made to ensure that the pAA block obtained by polymerization is less hydrophilic than the pSar block. The pAA block can be qualified as a hydrophobic block.

Preferably, the pAA block contains a chain from 8 to 120 AA units, preferably from 8 or 9 to 110, preferably from 12 to 100, and more preferably from 15 to 95.

In one preferred embodiment, the pAA block contains a chain from 8 to 50 AA units, preferably from 9 to 45, preferably from 12 to 40 and more preferably from 15 to 35 or even from 20 to 25.

In another preferred embodiment, the pAA block contains a chain from 50 to 120 AA units, preferably from 55 to 110, preferably from 60 to 100 and more preferably from 65 to 95.

In a preferred embodiment, the linear copolymer comprises:
a polysarcosine block, pSar, containing from 50 to 99 sarcosine units, preferably from 55 to 95, preferably from 60 to 85 and more preferably from 65 to 80; and
a poly(amino acid) block, pAA, containing from 8 to 50 AA units, preferably from 9 to 45, preferably from 12 to 40 and more preferably from 15 to 35 or even from 20 to 25.

In a preferred embodiment, the linear copolymer comprises:
a polysarcosine block, pSar, containing from 50 to 99 sarcosine units, preferably from 55 to 95, preferably from 60 to 85 and more preferably from 65 to 80; and
a poly(amino acid) block, pAA, containing from 50 to 120 AA units, preferably from 55 to 110, preferably from 60 to 100 and more preferably from 65 to 95.

In a preferred embodiment, the linear copolymer comprises:
a polysarcosine block, pSar, containing from 15 to 50 sarcosine units, preferably from 18 to 45, preferably from 22 to 40 and more preferably from 25 to 35; and
a poly(amino acid) block, pAA, containing from 8 to 50 AA units, preferably from 9 to 45, preferably from 12 to 40 and more preferably from 15 to 35 or even from 20 to 25.

In a preferred embodiment, the linear copolymer comprises:
a polysarcosine block, pSar, containing from 15 to 50 sarcosine units, preferably from 18 to 45, preferably from 22 to 40 and more preferably from 25 to 35; and
a poly(amino acid) block, pAA, containing from 50 to 120 AA units, preferably from 55 to 110, preferably from 60 to 100 and more preferably from 65 to 95.

Advantageously, the copolymer comprises two or more blocks wherein at least one block is pSar and at least one block is pAA. Preferably, the copolymer comprises two blocks wherein one block is pSar and one block is pAA.

Advantageously, the copolymer according to the invention has a hydrophilic fraction f(pSar) ranging from 5 to 80%, preferably from 10 to 70%, preferably from 15 to 60% and more preferably from 20 to 50%. The hydrophilic fraction f(pSar) can also be ranging from 5 to 60% or from 10 to 55%. The hydrophilic fraction, f(pSar), is the ratio in percentage of the number average molar mass (Mₙ) of the pSar block on the Mₙ of the copolymer.

Advantageously, the copolymer has a hydrophobic fraction f(pAA) ranging from 20 to 90%, preferably from 30 to 88%, preferably from 40 to 85% and more preferably from 50 to 80%. The hydrophobic fraction is the percentage of the Mₙ of the hydrophobic pAA block on the Mₙ of the copolymer.

Advantageously, the copolymer according to the invention has a number average molar mass, Mₙ, ranging from 500 g/mol to 50 000 g/mol, preferably from 800 to 45 000 g/mol, preferably from 1 000 to 40 000 g/mol, preferably from 1 200 to 35 000 g/mol and more preferably from 1400 to 32 000 g/mol.

Advantageously, the copolymer according to the invention has a number average molar mass, Mₙ, ranging from 4 500 g/mol to 50 000 g/mol, preferably from 4 800 to 40 000 g/mol, preferably from 5 200 to 30 000 g/mol, preferably from 5 500 to 25 000 g/mol and more preferably from 6 000 to 20 000 g/mol.

Advantageously, the linear copolymer according to the invention has a dispersity, D, ranging from 1 to 2, preferably from 1.0 to 1.8 and more preferably from 1.0 to 1.6.

Advantageously, the pAA block has a H hydrophobicity coefficient equal or higher than −0.50, preferably equal or higher than −0.25, preferably equal or higher than 0.00, preferably equal or higher than 0.50 and more preferably equal or higher than 0.80. Typically, pAA block has a H coefficient ranging from −0.50 to 20.00, preferably from −0.25 to 18.00, preferably from 0.00 to 15.00 and more preferably from 0.50 to 10.00 or even from 0.80 to 6.00. The H hydrophobicity coefficient of pAA block is determined by the equation E1 or E2 as defined above.

The hydrophobic pAA block comprises, preferably consists of, natural or unnatural amino acids units, AA units, preferably as long as AA units are hydrophobic compared to the Sar unit of the pSar block. According to the invention, the pAA block does not comprise sarcosine units.

Advantageously, amino acids of pAA block are D- or/and L- wherein D- refers to D isomer and L-refers to L isomer of amino acids. Preferably, amino acids of the pAA block are a mixture of D- and L- or only L- amino acids, or only D-amino acids. Preferably, when amino acids are a D-/L-mixture, the molar ratio between D-/L- is ranging from 30/70 to 70/30, preferably from 40/60 to 60/40, preferably from 45/55 to 55/45 and more preferably is 50/50.

Advantageously, AA of pAA is selected from the group comprising hydrophobic amino acid, protected hydrophobic amino acid, protected hydrophilic amino acid and combinations thereof. Hydrophobic amino acid is preferably selected from the group comprising alanine, valine, norleucine, leucine, isoleucine, methionine, phenylalanine, tryptophan, proline, tyrosine and combinations thereof.

Advantageously, AA of pAA is an amino acid with a lateral chain bearing at least an aryl and/or an heteroaryl group. The aryl and/or an heteroaryl group can be originally included in the lateral chain of the amino acids and/or added as a protected groups in the lateral chain of the AA. For example, the AA can be a glutamic acid protected with a benzyl group.

Without being bound by any theory, it is believed that aryl and/or heteroaryl group can participate and facilitate the encapsulation of hydrophobic compound, such as hydrophobic API, by the linear copolymer of the invention.

Functional groups on the lateral chains of AA can be protected by different protecting groups. Preferably, the protecting groups comprise at least an aryl and/or an heteroaryl group.

Functional groups such as hydroxyl groups, amine groups, aldehyde groups and carboxylic acids groups, of amino acids lateral chain might be protected by esters, carbonates, sulfonates allyl esters, ethers, silyl ethers, alkyl ethers, arylalkyl ethers, alkoxyalkyl ethers. The esters used to protect hydroxyl groups according to the invention can be formates, acetates, proprionates, butanoates, pentanoates, crotonates, benzoates, benzoyle formate, chloroacetate, trifluoroacetate, methoxyacetate, triphenylmethoxyacetate, p-chlorophenoxyacetate, 3-phenylproprionate, 4-oxopentanoate, 4,4-(ethylenedithio)pentanoate, pivaloate (trimethylacetate), crotonate, 4-methoxy-crotonate, benzoate, p-benzylbenzoate, 2,4,6-trimethylbenzoate.

Advantageously, when carbonates are used to protect the hydroxyl group, said carbonates are chosen among 9-fluorenylmethyl, ethyl, 2,2,2-trichloro-ethyl, 2-(trimethylsilyl)ethyl, 2-(phenylsulfonyl)ethyl, vinyl, allyl, and p-nitrobenzyl carbonate.

Advantageously, when silyl ethers are used to protect the hydroxyl group, said silyl ethers are chosen among trimethylsilyl, triethylsilyl, tert-butyldimethylsilyl, tert-butyldiphenylsilyl, triisopropylsilyl ether, and other trialkylsilyl ethers.

Advantageously, when alkyl, alkoxyalkyl and arylalkyl ethers are used to protect the hydroxyl group; alkyl ethers are chosen among methyl, benzyl, p-methoxybenzyl, 3,4-dimethoxybenzyl, trityl, tert-butyl, and allyl ether, or their derivatives; Alkoxyalkyl ethers which include acetals are chosen among methoxymethyl, methylthiomethyl, (2-methoxyethoxy)methyl, benzyloxymethyl, beta-(trimethylsilyl)ethoxymethyl, and tetrahydropyran-2-yl ether; Arylalkyl ethers are chosen among benzyl, p-methoxybenzyl (MPM), 3,4-dimethoxybenzyl, o-nitrobenzyl, p-nitrobenzyl, p-halobenzyl, 2,6-dichlorobenzyl, p-cyanobenzyl, 2- and 4-picolyl ethers.

Advantageously, amine group of AA lateral chain can be protected by arylalkylamines, carbamates, allyl amines, amides, and their derivatives, including tert-butyloxycarbonylamino (—NHBOC), ethyloxycarbonylamino, methyloxycarbonylamino, trichloroethyloxycarbonylamino, allyloxycarbonylamino (—NHAlloc), benzyloxocarbonylamino (—NHCbz), allylamino, benzylamino (—NHBn), fluorenylmethylcarbonyl (—NHFmoc), formamido, acetamido, chloroacetamido, dichloroacetamido, trichloroacetamido, phenylacetamido, trifluoroacetamido, benzamido, tert-butyldiphenylsilyl.

Advantageously, aldehyde groups of AA lateral chain can be protected with acyclic acetals, hydrazones, imines, and more specifically with dimethyl acetal, diethyl acetal, diiso-propyl acetal, dibenzyl acetal, bis(2-nitrobenzyl) acetal, 1,3-dioxanes, 1,3-dioxolanes, semicarbazones, and their derivatives.

Advantageously, carboxylic acids groups of AA lateral chain can be protected with optionally substituted C₁-C₆ aliphatic esters, optionally substituted aryl esters, silyl esters, activated esters, amides, hydrazides, such as and not limited to, ethyl, propyl, isopropyl, butyl, isobutyl, benzyl, and phenyl ester, wherein each group is optionally substituted. Additional protected carboxylic acids include oxazolines and ortho esters.

Those skilled in the art would know that the hydrophilicity of AA can be transformed from hydrophilic into hydrophobic using protecting groups on the functional groups on the lateral chains of AA.

Hydrophilic AA, which can be later protected to become hydrophobic includes cysteine, tyrosine, serine, threonine, aspartic acid, glutamic acid, asparagine, lysine, histidine, arginine, glycine and glutamine.

Protecting groups are preferably selected from alkyl, alkoxyalkyl and arylalkyl ethers such as trityl, tert-butyl, benzyl; esters such as trifluoroacetate; arylalkylamines, carbamates, allyl amines, amides, and their derivatives, including tert-butyloxycarbonylamino; acetates.

Protected hydrophilic AA, preferably, includes β-trityl-asparagine, β-benzyl-aspartate, S-benzyl-cysteine, cyclohexylglycine, γ-benzyl-glutamate, γ-tert-butyl-glutamate, ε-trifluoroacetyl-lysine, ε-Boc-lysine, ε-benzyl-lysine, β-benzyl-serine, β-acetyl-tyrosine, O-benzyl-tyrosine. A preferred protected hydrophilic AA is γ-benzyl-glutamate.

Preferably, the linear copolymer is chosen among copolymer of formula I or II,

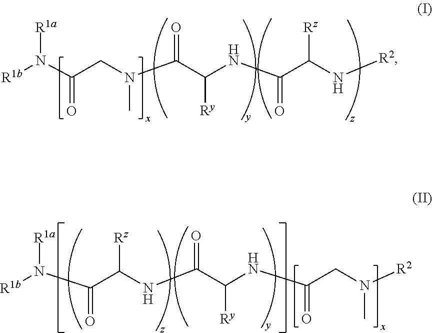

wherein
x is the number of sarcosine constitutional unit and is an integer ranging from 15 to 99,
y+z is the number of amino acid constitutional units and is an integer ranging from 8 to 120,
groups R^{y} and R^{z} are independently chosen from an amino acid lateral-chain group,
groups R^{1a} and R^{1b} are independently chosen from H, alkyl, alkenyl, alkynyl, cycloalkyl, aryl and heteroaryl,
group R² is chosen from H and a nitrogen protecting group.

When the amino acid comprises a cyclic lateral-chain group encompassing, then the group R^{y} and/or R^{z} is also linked to the N atom of the constitutional unit. Then the hydrogen atom represented in formula (I) and (II) is naturally lacking.

“x” is the total number of sarcosine constitutional units in pSar block. Preferably, x is an integer ranging from 20 to 95, preferably from 24 to 85 and more preferably from 28 to 80.

In one preferred embodiment, x is an integer ranging from 50 to 99 sarcosine units, preferably from 55 to 95, preferably from 60 to 85 and more preferably from 65 to 80.

In another preferred embodiment, x is an integer ranging from 15 to 50 sarcosine units, preferably from 18 to 45, preferably from 22 to 40 and more preferably from 25 to 35.

“y+z” is the total number of amino acid constitutional units. Preferably, y+z is an integer ranging from 8 or 9 to 110, preferably from 12 to 100 and more preferably from 15 to 95.

In one preferred embodiment, y+z is an integer ranging from 8 to 50, preferably from 9 to 45, preferably from 12 to 40 and more preferably from 15 to 35 or even from 2.0 to 25.

In one preferred embodiment, y+z is an integer ranging from 50 to 120, preferably from 55 to 110, preferably from 60 to 100 and more preferably from 65 to 95.

Advantageously,
x is ranging from 50 to 99, preferably from 55 to 95, more preferably from 60 to 85, even more preferably from 65 to 80, and
y+z is ranging from 8 to 50, preferably from 9 to 45, more preferably from 12 to 40, even more preferably from 20 to 25.

Advantageously,
x is ranging from 50 to 99, preferably from 55 to 95, more preferably from 60 to 85, even more preferably from 65 to 80, and
y+z is ranging from 50 to 120, preferably from 55 to 110, more preferably from 60 to 100, even more preferably from 65 to 95.

Advantageously, when x is ranging from 50 to 99, y+z is ranging from 50 to 120. Advantageously, when x is ranging from 55 to 95, y+z is ranging from 55 to 110. Advantageously, when x is ranging from 60 to 85, y+z is ranging from 60 to 100. Advantageously, when x is ranging from 65 to 80, y+z is ranging from 65 to 95.

Advantageously,
x is ranging from 15 to 50, preferably from 18 to 45, more preferably from 22 to 40, even more preferably from 25 to 35, and
y+z is ranging from 8 to 50, preferably from 9 to 45, more preferably from 12 to 40, even more preferably from 15 to 35, even more preferably from 20 to 25.

Advantageously, when x is ranging from 15 to 50, y+z is ranging from 8 to 50. Advantageously, when x is ranging from 18 to 45, y+z is ranging from 9 to 45. Advantageously, when x is ranging from 22 to 40, y+z is ranging from 12 to 40. Advantageously, when x is ranging from 25 to 35, y+z is ranging from 15 to 35 or even from 20 to 25.

Advantageously,
x is ranging from 15 to 50, preferably from 18 to 45, more preferably from 22 to 40, even more preferably from 25 to 35, and
y+z is ranging from 50 to 120, preferably from 55 to 110, more preferably from 60 to 100, even more preferably from 65 to 95.

“y” is the number of AA units in pAA sub-block comprised in the pAA block. Preferably, “y” is a number ranging from 0 to 120, preferably 0 to 110, preferably from 5 to 100, preferably from 9 to 95, preferably from 10 to 90, preferably from 15 to 85, preferably from 18 to 80, preferably from 20 to 75, preferably from 25 to 70, and more preferably from 30 to 65.

“z” is the number of AA units in pAA sub-block comprised in the pAA block. Preferably, “z” is a number ranging from 0 to 120, preferably 0 to 110, preferably from 5 to 100, preferably from 9 to 95, preferably from 10 to 90, preferably from 15 to 85, preferably from 18 to 80, preferably from 20 to 75, preferably from 25 to 70, and more preferably from 30 to 65.

Advantageously, y is a number ranging from 5 to 50 and z is a number ranging from 0 to 50. Preferably, y is ranging from 9 to 45 and z from 0 to 45. Preferably, y is ranging from 9 to 40 and z from 5 to 45. Preferably, y is ranging from 10 to 40 and z from 9 to 45. Preferably, y is ranging from 10 to 40 and z from 10 to 40.

In one preferred embodiment, y or z is 0. Preferably, z is 0 and y is ranging from 8 to 120, preferably from 9 to 110, preferably from 12 to 100, preferably from 15 to 95. In one preferred embodiment, z is 0 and y is an integer ranging from 8 to 50, preferably from 9 to 45, preferably from 12 to 40 and more preferably from 15 to 35. In one preferred embodiment, z is 0 and y is an integer ranging from 50 to 120, preferably from 55 to 110, preferably from 60 to 100 and more preferably from 65 to 95.

Advantageously, the pAA block has a H hydrophobicity coefficient equal or higher than −0.50, preferably equal or higher than −0.25, preferably equal or higher than 0.00, preferably equal or higher than 0.50 and more preferably equal or higher than 0.80. Typically, pAA block has a H coefficient ranging from −0.50 to 20.00, preferably from −0.25 to 18.00, preferably from 0.00 to 15.00 and more preferably from 0.50 to 10.00 or even from 0.80 to 6.00. The H hydrophobicity coefficient of pAA block is determined by:
the equation E1 as defined above when one of y or z is 0, with “n” corresponding to “y” when z=0 or to “z” when y=0; or
the E2 as defined above when both y and z are different from 0.

In formula (I) and (II), lateral chains of AA units of pAA block are represented by the group R^{y} and R^{z}. R^{y} and R^{z} groups are the lateral chains of AA units defined above. According to the invention, R^{y} and R^{z} are not H.

Advantageously, the unit bearing R^{y} and R^{z} groups of pAA block are independently chosen among natural or unnatural amino acids as long as the lateral chains of said hydrophobic pAA block are hydrophobic.

Preferably, R^{y} and/or R^{z} groups are bearing at least an aryl and/or an heteroaryl group. The aryl and/or an heteroaryl group can be on the amino acids and/or on the protecting group of the AA. For example, R^{y} and/or R^{z} groups can be the lateral chains of glutamic acid protected by a benzyl group, namely the lateral chain of γ-benzyl-D-glutamate.

Functional groups on the lateral chains of AA can be protected by different protecting groups, as disclosed above. Preferably, the protecting groups comprise at least an aryl and/or an heteroaryl group.

Alpha-carbons of the AA units which bear R^{y} and R^{z} groups can have a D- or L-configuration. In the present invention, when R^{y} or R^{z} is designated as L- or D-, preferably L-AA or D-AA, it means that the carbon bearing the group R^{y} or R^{z} is in L- or D-configuration. Preferably, when R^{y} or R^{z} is designated as L-AA or D-AA, it means that R^{y} or R^{z} is in L or D configuration of the corresponding AA.

Advantageously, alpha-carbons bearing R^{y} and R^{z} are independently in D- or L-configuration. Preferably, alpha-carbons bearing R^{y} and R^{z} have a different configuration. Preferably, R^{y} is the lateral chain of a hydrophobic L-AA, and R^{z} is the lateral chain of a hydrophobic D-AA. Preferably, R^{y} is the lateral chain of a hydrophobic D-AA, and R^{z} is the lateral chain of a hydrophobic L-AA.

Preferably, at least one of R^{y} and R^{z} is the lateral chain of an AA bearing at least an aryl and/or a heteroaryl group. In particular, both R^{y} and R^{z} are the lateral chain of an AA bearing at least an aryl and/or a heteroaryl group.

Preferably, R^{y} and R^{z} are independently chosen from —CH₃, —CH(CH₃)₂, —(CH₂)₃—CH₃, —CH₂—CH(CH₃)₂, —CH(CH₃)—CH₂—CH₃, —CH₂—CH₂—S—CH₃, —CH₂—C₆H₅, —CH₂-(1H-indol-3-yl), —CH₂—CH₂—CH₂—, —CH₂-4-(OH)C₆H₄, —CH₂—C(O)—NH—C(C₆H₅)₃, —CH₂—C(O)—OCH₂—C₆H₅, —CH₂—S—CH₂—C₆H₅, —C₆H₁₁, —CH₂—CH₂—C(O)—O—CH₂—C₆H₅, —CH₂—CH₂—C(O)—O—C(CH₃)₃, —(CH₂)₄—NH—C(O)—CF₃, —CH₂—O—CH₂—C₆H₅, —CH₂- 4-(acetyl)-C₆H₅, and —CH₂—C₆H4O—CH₂—C₆H₅.

Preferably, R^{y} and R^{z} are independently chosen from —CH₂—CH(CH₃)₂, —CH₂—C₆H₅, —CH₂-4-(OH)C₆H₄, —CH₂—CH₂—C(O)—O—CH₂—C₆H₅, —CH₂—CH₂—C(O)—O—C(CH₃)₃, and —C₆H₁₁.

Preferably, R^{y} and R^{z} are independently chosen from —CH₂—C₆H₅, —CH₂-4-(OH)C₆H₄, —CH₂—CH₂—C(O)—O—CH₂—C₆H₅, —CH₂—C(O)—NH—C(C₆H₅)₃, —CH₂—C(O)—OCH₂—C₆H₅, —CH₂—S—CH₂—C₆H₅, —CH₂—O—CH₂—C₆H₅, —CH₂-4-(acetyl)-C₆H₅, and —CH₂—C₆H4O—CH₂—C₆H₅.

Preferably, R^{y} and R^{z} are independently chosen from the lateral chain of L-leucine, L-phenylalanine, L-tyrosine, γ-benzyl-L-glutamate, γ-tert-butyl-L-glutamate, L-cyclohexylglycine, D-leucine, D-phenylalanine, D-cyclohexylglycine, D-tyrosine, γ-benzyl-D-glutamate, γ-tert-butyl-D-glutamate.

In some embodiments, each R^{y} is independently the lateral chain of L-leucine, L-phenylalanine, L-tyrosine, γ-benzyl-L-glutamate or γ-tert-butyl-L-glutamate. In some embodiments, each R^{z} is independently the lateral chain of D-leucine, D-phenylalanine, D-cyclohexylglycine, D-tyrosine, y-benzyl-D-glutamate, γ-tert-butyl-D-glutamate. Preferably, each R^{y} and R^{z} is independently —CH₂—CH₂—C(O)—O—CH₂—C₆H₅, —CH₂—CH₂—C(O)—O—C(CH₃)₃. Preferably, each R^{y} is independently the lateral chain of γ-benzyl-L-glutamate or γ-tert-butyl-L-glutamate. Preferably, each R^{z} is independentlythe lateral chain of γ-benzyl-D-glutamate, γ-tert-butyl-D-glutamate.

Preferably, R² is a nitrogen protecting group. Nitrogen protecting group can be chosen from any group well-known in the state of the art. Nitrogen protecting groups include, but are not limited to arylalkylamines, carbamates, allyl amines, amides, and their derivatives, including tert-butyloxycarbonylamino (—NHBOC), ethyloxycarbonylamino, methyloxycarbonylamino, trichloroethyloxycarbonylamino, allyloxycarbonylamino (—NHAlloc), benzyloxocarbonylamino (—NHCbz), allylamino, benzylamino (—NHBn), fluorenylmethylcarbonyl (—NHFmoc), formamido, acetamido, chloroacetamido, dichloroacetamido, trichloroacetamido, phenylacetamido, trifluoroacetamido, benzamido, tert-butyldiphenylsilyl.

Preferably, R^{1a} and R^{1b} are independently chosen from H, (C₁-C₁₀) alkyl, (C₂-C₁₀) alkene, (C₂-C₁₀) alkyne, (C₃-C₁₀) cycloalkyl, (C₅-C₁₀) aryl, (C₄-C₁₀) heteroaryl. Preferably, R^{1a} and R^{1b} are independently chosen from H, (C₁-C₆) alkyl, (C₂-C₈) alkene, (C₂-C₁₀) alkyne, (C₃-C₁₀) cycloalkyl, (C₅ or C₆) aryl, (C₄ or C₅) heteroaryl. More preferably, R^{1a} and R^{1b} are independently chosen from H, methyl, ethyl, n-propyl, iso-propyl, n-butyl, iso-butyl, sec-butyl, tert-butyl, n-pentyl, neopentyl, n-hexyl, cyclopropyl, cyclobutyl, cyclopentyl, cyclohexyl, and a group phenyl and/or benzyl optionally substituted with a halogen, —CH₃, —CF₃, —OCH₃, OH.

Preferably, R^{1a} is H, R^{1b} is neopentyl and R² is selected from a H or —C(O)CH₃.

Advantageously, the group NR₁ₐR^{1b} is a residue derived from the polymerization initiator linked to a first block.

Advantageously, the group R² is a residue derived from a terminating agent or a capping agent linked to the last block formed during the linear copolymer's synthesis.

According to a preferred embodiment of the invention, when the first block is pSar and the last block is pAA, the linear copolymer is defined by general formula I, wherein
groups R^{1a} and R^{1b} are independently chosen from H, an alkyl group or an aryl group, group R² is chosen from H, alkyl group and a group selected from R³OC(O)—, R³C(O)—, R³SO₂—,
wherein R³ is selected from (C₁-C₁₀) alkyl, (C₂-C₁₀) alkene, (C₂-C₁₀) alkyne, (C₃-C₁₀) cycloalkyl, (C₅-C₁₀) aryl and (C₄-C₁₀) heteroaryl.

According to a preferred embodiment of the invention, when the first block is pAA and the last block is pSar, the linear copolymer is defined by general formula II, wherein
groups R^{1a} and R^{1b} are independently chosen from H, an alkyl group or an aryl group,
group R² is chosen from H, alkyl group and a group selected from R³OC(O)—, R³C(O)—, R³SO₂—,
wherein R³ is selected from (C₁-C₁₀) alkyl, (C₂-C₁₀) alkene, (C₂-C₁₀) alkyne, (C₃-C₁₀) cycloalkyl, (C₅-C₁₀) aryl and (C₄-C₁₀) heteroaryl.

Preferably, the group R² in formula I or II is a residue derived from a capping agent selected from an alkyl group and a group selected from R³OC(O)—, R³C(O)—, R³SO₂—. Preferably, R³ is selected from H, (C₁-C₆) alkyl, (C₂-C₈) alkene, (C₂-C₁₀) alkyne, (C₃-C₁₀) cycloalkyl, (C₅ or C₆) aryl, (C₄ or C₅) heteroaryl. More preferably R³ is selected from methyl, ethyl, propyl, butyl, trifluoromethyl.

Preferably, the copolymer is selected from copolymers C1 to C12 of the examples represented by formulas (I′) or (II′)

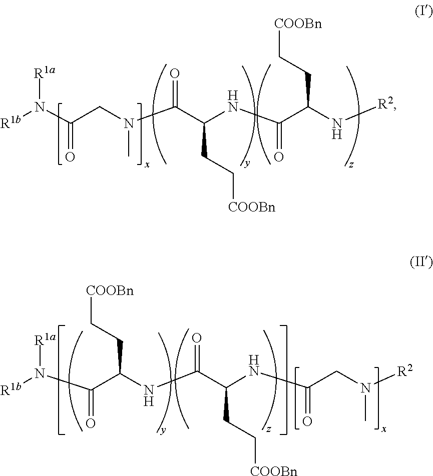

wherein
R^{1a} is H and R^{1b} is —CH₂—C(CH₃)₃
R² is H or CH₃C(O)—, and
x and “y+z” have different values depending on the copolymers according to the invention described in examples below.

### Method of Preparation of the Copolymer

The present invention also relates to a method of preparation of linear copolymer. Preferably, linear copolymers are synthesized by polymerization of sarcosine derivates and amino acid derivates. Preferably, copolymers are synthesized by ring opening polymerization in which Sar and AA derivates are the respective N-carboxyanhydrides (NCAs) or thiocarboxyanhydrides (NTAs) of the corresponding constitutional units.

Advantageously, the synthesis of linear copolymers according to the invention having a limited number of constitutional units of Sar and/or AA is facilitated and thus less expensive compared to the prior art. Industrial scale-up of the preparation of the linear copolymers according to the invention is also facilitated.

According to the invention, Sar and AA derivates correspond to compounds represented respectively by formulas III and IV as follows

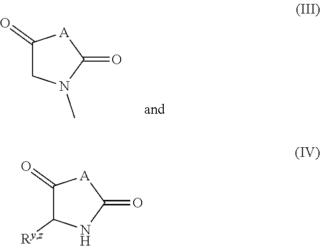

wherein A is O or S; and R^{y}, R^{z} are as described above.

Compounds of formula III and IV also called derivates, are NCAs when A is O and NTAs when A is S.

Compound of formula III can be the monomer used for preparing the pSar block. It is also designated as NCA/NTA of sarcosine or sarcosine NCA/NTA or Sar NCA/NTA.

Compound of formula IV can be the monomer used for preparing the pAA block. Compound IV is designated as IV^{y} when it is used to prepare units of pAA block having the superscript y. Similarly, compound IV^{z} is used to prepare units of pAA block having the superscript z. R^{y} and R^{z} are as described above. It is also designated as NCA/NTA of amino acid or amino acid NCA/NTA or AA NCA/NTA.

The linear copolymer of the present invention can be prepared by the method comprising
a step a) of formation of a first block by ring opening polymerization by reacting a first derivate in the presence of a polymerization initiator, wherein the first derivate is chosen from compound III or at least one compound IV,
an optional step b) of isolation of the first block formed in step a),
a step c) of formation of a last block by mixing a second derivate chosen from compound III or at least one compound IV, with the first block obtained in step a) or isolated in the optional step b),
   wherein the first and the second derivates are different and one of them is compound III,
a step d) of recovery of the copolymer.

Advantageously, the step d) of recovery of the linear copolymer of the invention comprises the isolation and purification of the copolymer resulting from step c). Preferably, the step of recuperation d) is carried out after the consumption of the derivate introduced in step c).

Advantageously, when the last block is formed by addition of the derivate into the mixture obtained in step a), the step c) is carried out after the consumption of the derivate introduced in step a).

Advantageously, the derivate other than sarcosine introduced in step a) or c) is selected from two AAs having the formula IV. Preferably, one of amino acids acid has the formula IV^{y} with R^{y} group and the other AA has the formula IV² with R^{z} group. Carbon atom bearing R^{y} and R^{z} can be in a D- or L-configuration.

Advantageously, the copolymer can be prepared by one-pot method wherein the first block formed in step a) is not isolated in the optional step b). In this embodiment, the last block is formed by directly mixing the second derivate into the mixture obtained in step a) and after the consumption of the derivate introduced in step a).

In an embodiment, the derivate introduced in step a) is a compound III and the derivate introduced in step c) is a compound IV.

In another embodiment, the derivate introduced in step a) is a compound IV and the derivate introduced in step c) is a compound III.

Preferably, step a) and/or c) is/are carried out under reduced pressure or at atmospheric pressure.

Preferably, the step a) and/or c) is/are carried out at a temperature ranging from 0 to 80° C., preferably from 5 to 60° C., preferably from 10 to 50° C., preferably from 15 to 40° C., preferably from 20 to 30° C. and more preferably from 22 to 28° C.

Advantageously, step a) and/or c) are carried in a solvent selected from N,N-dimethylformamide, tetrahydrofuran, dimethylsulfoxide, water, acetonitrile, ethyl acetate. Preferably, the solvent is N,N-dimethylformamide.

Preferably, the solvent of step a) is identical to the solvent of step c).

In a preferred embodiment, derivates are introduced in step a) and/or c) as solid powders.

In some embodiments, derivates are introduced in step a) and/or c) as solutions in a solvent.

Preferably, the solvent of the derivates solution is identical to the solvent used for the reaction.

Preferably, the polymerization initiator is an amine preferably having the formula (R^{1a})(R^{1b})NH, wherein R^{1a} and R^{1b} are as described above.

Preferably, the molar ratio of the first derivate to polymerization initiator is ranging from 5 to 120, preferably from 20 to 110, preferably from 30 to 100 and more preferably from 40 to 95.

Advantageously, the method of preparation of the copolymer can also comprise a step of termination performed prior to step d) after the consumption of the derivate introduced in step c). Preferably, the step of termination corresponds to the consumption of all the derivate introduced in step c). Advantageously, the step of termination can be carried out with a terminating agent well known in the art. Preferably, the step of termination is performed by protonation. Then, R² is preferably H. Optionally, the terminating agent can be selected from water, an acid such as acetic acid, triflic acid or trifluoroacetic acid.

Preferably, the method of preparation comprises a step of capping carried out prior or after step d) by the addition of a capping agent and optionally any other reagents required for the reaction. When the capping step is prior to step d), the introduction of the capping agent is carried out after the consumption of the derivate introduced in step c). In this embodiment, the capping step can be carried out after the termination step. In this embodiment, capping agent can also be a terminating agent. Then, termination step and capping are performed in a single step.

When the capping step is after step d), said capping step is carried out by mixing the copolymer with the capping agent in presence of a solvent as defined above.

In any case, preferably, the capping step is carried out at a temperature ranging from 0 to 80° C., preferably from 5 to 60° C., preferably from 10 to 50° C. and more preferably from 15 to 40° C., preferably from 20 to 30° C. and more preferably from 22 to 28° C.

Advantageously, the capping step is performed at the same temperature than step a) and/or c). The capping step is preferably performed by addition of a capping agent having the formula R²X. R² is as described above, and X is a leaving group. Preferably, X is a leaving group well-known from the skilled person. Preferably, X is selected from Cl—, I—, Br—, CF₃C(O)—, CH₃C(O)—.

### Nanoparticles

One other aspect of the invention is directed to nanoparticles comprising linear copolymers as defined above. Nanoparticles are formed by self-assembly of linear copolymers according to the invention.

Advantageously, nanoparticles comprise linear copolymers and at least one active compound.

According to one aspect of the invention, nanoparticles according to the invention have a hydrodynamic diameter, Dₕ, lower than 900 nanometers (nm), preferably lower than 600 nm, preferably ranging from 1 to 600 nm, preferably from 2 to 500, preferably from 5 to 300 nm, preferably from 8 to 200 nm and more preferably from 10 to 110 nm.

Preferably, the nanoparticles have a polydispersity index, PDI, lower than 0.7, preferably ranging from 0.02 to 0.70, preferably from 0.05 to 0.60 and more preferably from 0.10 to 0.50.

Advantageously, the active compound can be bound by non-covalent interactions with the nanoparticles. Preferably, the active compound can be bound within the nanoparticle or absorbed on its surface by non-covalent interactions. Preferably, the active compound is bound with the nanoparticle by hydrophobic interactions.

Preferably, the active compound is hydrophobic. The compound can be active pharmaceutical ingredients (APIs) and nucleic acids such as siRNA, miRNA.

According to one preferred embodiment, the nanoparticles comprise at least two active compounds, preferably at least two APIs.

Preferably, the hydrophobic compound is loaded within the nanoparticle.

Hydrophobic compound, preferably hydrophobic API, is a compound having a water-solubility of less than 10 g/L.

The nanoparticles comprising copolymers and API are called drug-loaded nanoparticles.

Advantageously, drug-loaded nanoparticles according to the invention have at least one of the following advantages:
preventing or delaying the API degradation,
controlling the API release,
improving the API bioavailability, and
for anti-tumor applications, enhancing the API absorption into tumor tissues by passive targeting through the enhanced permeation and retention (EPR effect).

Preferably, APIs are chosen to be suitable for targeting a disease selected by those skilled in the art. Examples of APIs include anticancer agents, antimicrobial agents, antiviral agents, anti-inflammatory agents, immunosuppressive drugs, steroid drugs, hormone drugs, and anti-angiogenic agents. These drug molecules may be used singly or in combination of two or more thereof.

Preferably, API is selected from 10,11-methylenedioxycamptothecin, 10-hydroxy-7-ethylcamptothecin (SN38), 2-chloroadenosine trimetrexate, 5-azacytosine, 5-azadeoxycytosine, 5′-deoxyfluorouridine, 5-fluorouracil (5-FU), 6-mercaptopurine, 6-thioguanine, 9-aminocamptothecin, 9-nitrocamptothecin, acyclovir, aldesleukin, allopurinol, amantadine, amiodarone, aminopterin, amsacrine, asparaginase, bleomycin, budesonide, busulfan, camptothecin, capecitabine, carboplatin, celecoxib, CI-973, cisplatin, cladribine, CPT-11, curcumine, cyclophosphamide, cyclosporin, cytarabine, dacarbazine, daunorubicin, deoxycytidine, docetaxel, doxorubicin, eniluracil, epirubicin, epothilones A-E, etoposide, etoposide phosphate, etoposide phosphate, florafur, ganciclovir, gemcitabine (gemzar), ifosphamidemefosphamide, irinotecan, JM-216, karenitecin, karenitecin, lamivudine, L-phenylalanine mustard, methotrexate, methylene-10-deazaminopterin (MDAM), mitomycin, mitoxantrone, ormaplatin, oxaplatin, paclitaxel, perfosfamide, picoplatin, platinum-DACH, procarbazine, rapamycin, resveratrol, rimantadine, satraplatin, semustine, tamoxifen, TAS 103, temozolomide, tetraplatin, tomudex, topotecan, tretinoin, trophosphamide carmustine, UFT, valacyclovir, vinblastine, vincristine, vindesine, vinorelbine, zidovudine and combinations or a pharmaceutically acceptable salt thereof.

One aspect of the invention is the use of drug-loaded nanoparticles as a drug product, preferably in a cancer treatment. One preferred aspect of the invention is a method of treating or preventing a disease or a condition, such as cancer, comprising the administration to a patient in need thereof nanoparticles according to the invention. Another preferred aspect of the invention is the use of nanoparticles according to the invention for the manufacture of a drug product for a therapeutic application, preferably for the treatment of cancer. In particular, the hydrophobic API can be a cytotoxic agent, such as a taxoid and more particularly paclitaxel.

Preferably, the hydrophobic API is advantageously an anticancer drug, more preferably chosen among paclitaxel (taxol), doxorubicin, daunorubicin, vinblastine, docetaxel (taxotere), 10-hydroxy-7-ethylcamptothecin (SN38).

Advantageously, nanoparticles improve the solubility of hydrophobic APIs. Water-solubility represents the concentration at which a compound is soluble in water. For a hydrophobic API loaded in nanoparticles, the water solubility of the API is considered to be the API concentration, [API], in the suspension of nanoparticles although it is not technically speaking in solution. Advantageously, nanoparticles improve the water solubility of hydrophobic APIs by a factor greater than 100, preferably greater than 200, preferably greater than 500, preferably greater than 1000, preferably greater than 2000 and more preferably greater than 3000.

Preferably, the API in API-loaded nanoparticles has a water solubility, noted [API], from 0.2 to 2.0 g/L, preferably from 0.3 to 1.5 g/L, preferably from 0.4 to 1.5 g/L and more preferably from 0.5 to 1.5 g/L.

Advantageously, a high value of [API] permits to lower the volume of particle needed to be effective against the API's target such as a tumor.

### Method of Preparation of Nanoparticles

Copolymers according to the invention can spontaneously self-assemble in aqueous solutions into nanoparticles presenting a corona formed by the pSar block and a core formed by the pAA block.

The invention concerns a method of preparation of nanoparticles comprising linear copolymers as described above. Preferably, the nanoparticles are obtained by a method comprising the following steps:
preparing an organic solution containing an organic solvent and the linear copolymer of the invention,
then mixing the organic solution with an aqueous solution,
then removing the organic solvent.

The organic solvent is preferably a polar aprotic solvent.

The invention is also directed to a method of preparation of nanoparticles comprising linear copolymer and an active compound, the copolymer and the active compound being as described above.

In the following, the method of preparation of nanoparticles is described for hydrophobic API but said method applies also for any hydrophobic compound as described above.

Advantageously, if the formation of nanoparticles is performed in presence of a hydrophobic API, it permits the encapsulation of said API in the hydrophobic core of the nanoparticle.

The encapsulation of the hydrophobic API by said nanoparticles can occur with a method comprising the following steps:
preparing an organic solution containing organic solvent, the copolymer as described above and at least one hydrophobic API as described above,
mixing the organic solution with an aqueous solution,
optionally removing the organic solvent,
recovering the nanoparticles.

A preferred method of preparing nanoparticles comprises:
a step i) of mixing a hydrophobic API and a copolymer according to the invention in an organic solvent in order to obtain an organic solution,
a step ii) of mixing the solution obtained in step i) with an aqueous solution upon stirring in order to obtain nanoparticles,
an optional step iii) of filtration of the nanoparticles,
an optional step iv) of purification, for example by gel filtration chromatography,
an optional step v) of filtration of the nanoparticles,
an optional step vi) of freezing or freeze-drying,
a step vii) of recovery of the nanoparticles.

In an embodiment, step ii) is carried out by adding the organic solution obtained in step i) into an aqueous solution. In another embodiment, step ii) is carried out by adding the aqueous solution into the solution obtained in step i).

Preferably, the organic solvent is a polar aprotic solvent. Preferably, the polar aprotic solvent is selected from N,N-dimethylformamide and N,N-dimethylacetamide.

In a preferred embodiment, the solvent used in step ii) is identical to the solvent used in step a) and/or c) of the preparation of the copolymer. This embodiment facilitates the purification of the copolymers and limits the potential effect of the residual solvent coming from the preparation of the copolymer on the self-assembly into nanoparticles. Preferably, the solvent is N,N-dimethylformamide.

Preferably, the aqueous solution is water or comprises water as the main solvent.

In a preferred embodiment, wherein nanoparticles are suitable as a pharmaceutical dosage form, the aqueous solution of step ii) can further comprises a cryo-protectant and/or a buffer.

The cryo-protectant prevents the damage or alteration of other compounds related to freezing or freeze-drying and allows to reach a physiological osmolarity. Cryo-protectant includes, but is not limited to: monosaccharides, disaccharides, polyalcohols, amino acids, glycine, polyvinyl pyrrolidine, polyethylene glycol, mannitol, sorbitol, sucrose, glucose, raffinose, sucralose, lactose, trehalose, dextran, and dextrose. Preferably, cryo-protectant is trehalose.

The buffer allows to reach a physiological pH and also has an impact on osmolarity. The buffer includes, but is not limited to: a phosphate buffer, a phosphate buffer saline (PBS), a histidine buffer or HEPES buffer.

Advantageously, the mixing conditions in step ii) are modulated to optimize the resulting nanoparticles. Said conditions are for example the nature of mixing (magnetic stirring, homogenizer), the mixing time, the order of addition of one solution to the other or the flow rate of addition.

The optional step iii) can be performed to remove unloaded (free) API from the suspension in order to start the purification process and preserve the life time of the column used in optional step iv).

The optional step iv) can be performed to obtain a nanoparticle suspension in aqueous solution by removing the polar protic solvent and unloaded API. When the aqueous solution is water, then step iv) allow obtention of a nanoparticle suspension in pure aqueous solution.

Preferably, a filtration at step v) is carried out with a 0.2 μm, 0.22 μm or 0.45 μm sterile filter in order to sterilize the nanoparticles.

Advantageously, nanoparticles according to the invention have a loading efficiency, LE, of more than 20%, preferably more than 30%, preferably more than 40%, preferably more than 45%, preferably more than 50%, preferably more than 55%, preferably more than 60%, preferably more than 65% and more preferably more than 70%. Advantageously, the loading efficiency is between 40 and 99%, preferably between 50 and 96%, preferably between 60 and 95% and more preferably between 70 and 94%.

The loading efficiency is the weight ratio of the mass of API loaded into nanoparticles on the mass of feeding API.

Advantageously, the feed weight ratio, FWR, is ranging from 1 to 100%, preferably from 2 to 80%, preferably from 3 to 60%, preferably from 4 to 50%, preferably from 5 to 40% and more preferably from 10 to 30%. FWR is the weight ratio of the mass of feeding API on the mass of feeding copolymer.

### EXAMPLES

In order that the disclosure described herein may be better understood, the following examples are provided. It is understood that these examples are for illustrative purposes only and should not be construed as limiting this disclosure in any manner.

In the following examples copolymers according to the invention comprise a block of poly(sarcosine) and a block derived from γ-benzyl-L-glutamate and/or γ-benzyl-D-glutamate.

### Materials and Methods

Paclitaxel was supplied by Key Organics. All the organic solvents (HPLC grade) were provided by VWR. Water was ultrapure grade. Unless stated otherwise, the temperature was room temperature (22-28° C.).

FTIR Spectroscopy

NCA polymerization was monitored by Fourrier-transform infrared (FTIR) spectroscopy on a Thermo Scientific Nicolet iS5 spectrometer equipped with an ID7 ATR module. Data were processed using OMNIC 9.7 software. Polymerizations were stopped when NCA-associated carbonyl bands at 1850 and 1778 cm⁻¹ had disappeared, corresponding to the total NCA consumption.

### PROTON NMR

Proton Nuclear Magnetic Resonance (¹H NMR) was performed on a 80 MHz MAGRITEK Spinsolve 80 Carbon, with the following parameters: NS=64, repetition time=10 to 30 s, pulse angle=90, by dissolving the product in deuterated dimethylsulfoxide (d₆-DMSO) at a concentration between 20 and 50 g/L. Data were processed using SpinSolve and MestReNova softwares.

### SEC

Size-exclusion chromatography (SEC) analyses were performed in DMF containing 0.45% w/v LiBr on a Agilent 1260 LC equipped with a diode array detector (UV-Vis) and a differential refractive index detector (dRI) and a three-column set of PSS GRAM analytical columns (100 Å, 8×300 mm, 10 μm; 100 Å, 8×300 mm, 10 μm; 1000 Å, 8×300 mm, 10 μm) with exclusion limits from 100 to 1000 000 g/mol. Analyses were carried out on samples prepared in DMF containing 0.45% w/v at 45° C. using DMF containing 0.45% w/v LiBr as eluent (1 mL/min). Data was acquired and processed with OpenLAB Chemstation software.

### SEC for the Characterization of Copolymers

To determine the number average molar mass (Mₙ) and dispersity (D) of polymers and copolymers, analyses were carried out on polymer samples prepared at 4 g/L. EasiVial kit of polystyrenes from Agilent was used as standard (266 to 66 000 g/mol). And data was further processed with Cirrus add-on.

### SEC for the Characterization of Nanoparticles

To determine the copolymer concentration in the PTX-loaded nanoparticles ([C in NPs]), a volume of 400 μL of each sample of NPs was dried using a centrifugal evaporator (SP Genevac EZ2, water evaporation automatic program). The samples injected in SEC were prepared by dissolving the dried sample in 1 mL of DMF containing 0.45% w/v LiBr, hence a dilution by 2.5.

A standard curve in DMF containing 0.45% w/v LiBr was prepared for each copolymer with polymer concentrations ([C]) between 1 and 4 g/L. Linear regression of the area obtained for the RI signal of the polymer versus [C] was plotted for each polymer to obtain the standard curve equation.

For each formulated sample, [C in NPs] was determined using the peak area obtained and the appropriate standard curve equation, and then multiplying by the factor of dilution.

Loading content (LC) of the PTX-loaded NPs was calculated as the ratio of the mass of PTX recovered at the final stage of the NPs' preparation (m(PTX in NPs), obtained by UPLC) divided by the mass of the NPs; the mass of NPs being the sum of the mass of copolymer (obtained using [C in NPs] by SEC) and m(PTX in NPs).

### DLS

DLS measurements were carried out on a Zetasizer Pro (Malvern Panalytical) equipped with a He—Ne laser (633 nm), at 25° C. and a scattering angle of 174.8°. Software used was ZS Explorer. A low volume plastic cell of 10 mm optical path length was filled with 70 μL of sample. Viscosity of the dispersant was corrected according to the solvent or mixture of solvents used. Data was acquired on three different measurements with an automatic optimization of the number and duration of runs per measurement. Results are expressed as an average of these 3 measurements. Dₕ of the objects is the intensity mean for each population. PDI is calculated from the autocorrelation functions using the cumulant method.

### UPLC

Ultra-performance liquid chromatography (UPLC) measurements were performed on an Acquity UPLC H-class from Waters equipped with a reversed-phase column (Acquity BEH, C18, 130 Å, 50 mm×2.1 mm×1.7 μm, Waters) and a diode array detector (DAD) Acquity eλ from Waters. Data was acquired and processed with Empower 3 software. The gradient elution was performed with 2 solvents: solvent A was water containing 0.05 vol % trifluoroacetic acid (TFA) and solvent B acetonitrile (ACN) containing 0.05 vol % TFA. Column was equilibrated for at least 30 min at a A:B mixture of 80:20. The sequence duration was 8 min with the following gradient of A:B solvents: 0-3 min 80:20, 3-5 min 5:95, 5-8 min 80:20. Eluents were degassed by the machine. Flow rate was 0.5 mL/min. Column temperature was 35° C. Injected volume was 3 μL. UV detector was set at 254 nm. Typical retention time of PTX was 2.24 min.

Loading efficiency (LE) of the PTX-loaded NPs was determined by UPLC. The injected samples were prepared by dissolving 90 μL of NPs in 910 μL of ACN:DMF 90/10 v/v, hence a dilution by 11.1.

A standard curve in ACN:(H₂O/DMF 90/10) 90:10 v/v was prepared with PTX concentrations ([PTX]) between 5 and 20 μg/mL. Linear regression of the area at 254 nm versus [PTX] was plotted to obtain the standard curve equation.

For each formulated sample, PTX concentration in nanoparticles ([PTX in NPs]) was determined using the peak area obtained and the standard curve equation, and then multiplying by the factor of dilution.

LE was calculated as the ratio of m(PTX in NPs) (obtained using [PTX in NPs]) divided by the mass of PTX initially fed in the NPs' preparation.

### HS-GC

Headspace gas chromatography (HS-GC) was used to dose the content of DMF as a residual solvent in formulations. HS-GC were performed on an Agilent 7890B GC system equipped with split/splitless injector, a flame ionization detector (FID), an Agilent 7697A autosampler and using a CP Sil 5CB column (50 m length, 0.32 mm diameter, 5 μm film thickness). A known quantity of sample was introduced into a 20 mL crimp vial and dissolved with a few milliliters of a low volatile solvent (water, DMSO or NMP). The vial was crimped and incubated in order to reach an equilibrium of concentration of the residual solvent between the liquid phase and the gas phase. The headspace of the vial was then injected into the GC system via a split injector and the residual solvent was detected by FID. Data were acquired and processed using OpenLAB Chemstation software.

### ABBREVIATIONS

The following abbreviations have been used in the examples:
[C] Copolymer concentration
[C in NPs] Copolymer concentration in the nanoparticles
[PTX] PTX concentration
[PTX in NPs] PTX concentration in the nanoparticles
¹H NMR Proton Nuclear Magnetic Resonance
Ð Dispersity
D-GluOBzl γ-benzyl-D-glutamate
Dₕ Hydrodynamic diameter
DLS Dynamic light scattering
DMAP Dimethylaminopyridine
DMF N,N-Dimethylformamide
DMSO Dimethylsulfoxide
eq Equivalent
f(pSar) Hydrophilic (polysarcosine) fraction
FTIR Fourrier-transform infrared
FWR Feed weight ratio
HS-GC Headspace gas chromatography
LC Loading content
LE Loading efficiency
L-GluOBzl γ-benzyl-L-glutamate
Mₙ Number average molar mass
MTBE Methyl tert-butyl ether
NCA N-carboxyanhydride
NMM N-Methylmorpholine
NP Nanoparticle
PDI polydispersity index
PES Polyethersulfone
pSar Polysarcosine
PTX Paclitaxel
Sar Sarcosine
SEC Size exclusion chromatography
TMS Tetramethylsilane
UPLC Ultra-performance liquid chromatography

In the following examples, in accordance with methods described above,
values of x, y+z for each copolymer and f(pSar) were determined by ¹H NMR;
Mₙ and Ð were determined by SEC;
Dₕ and PDI were determined by DLS;
[PTX] and LE were determined by UPLC;
LC was determined by SEC.

### Example 1: Preparation of PSAR Blocks

Sar NCA (4.34.10-2 mol, 75 eq) was dissolved in dry DMF at room temperature followed by the addition of neopentylamine as initiator (73 μL, 6.20.10-4 mol, 1 eq). The reaction mixture was stirred at room temperature and after the end of CO₂ evolution, completion of the reaction was confirmed by FTIR spectroscopy.

Precipitation of the polymer was performed at room temperature by pouring the reaction mixture on 300 mL of ethyl acetate upon vigorous stirring. After filtration, the product was reslurried with 2×100 mL of ethyl acetate and then dried under vacuum.

pSar block S1 with x=75 was obtained with a yield of 70%, Mₙ of 4570 g/mol and D of 1.16.

pSar block S2 was prepared following the same procedure except that 28 eq of Sar NCA (1.62.10-2 mol) was used. pSar block S2 with x=29 was obtained with a yield of 85%, Mₙ of 2200 g/mol and Ð of 1.22.

### Example 2: Preparation of Copolymers of Formula I

### Procedure for Two-Step Synthesis

pSar block S1 synthetized in example 1 (Mₙ=4570 g/mol, 1.31.10⁻⁴ mol, 1.0 eq) was dissolved in dry DMF (17 mL) at room temperature. A mixture of L-GluOBzl NCA (6.42.10⁻³ mol, 49 eq) and D-GluOBzl NCA (6.42.10⁻³ mol, 49 eq) powders was added to the reaction medium. The reaction mixture was stirred at 5° C. and after the end of CO₂ evolution, completion of the reaction was confirmed by FTIR spectroscopy.

Precipitation of the copolymer was performed at room temperature by pouring the reaction mixture on 200 mL of MTBE upon vigorous stirring. After filtration, the product was reslurried with 2×100 mL of MTBE and then dried under vacuum. Copolymer 1 was obtained with a yield of 76%.

Copolymers C2 to C5 according to the invention were prepared following the same procedure as C1 with the pSar block and quantities presented in Table 1.

TABLE 1
preparation conditions of copolymers C2 to C5.

| | | | | D- | L- | |
|---|---|---|---|---|---|---|
| Copol- | Yield | pSar | n of pSar | GluOBzl | GluOBzl | DMF |
| ymer | (%) | block | block (mol) | NCA (eq) | NCA (eq) | (mL) |
| C2 | 84 | S1 | 4.40.10⁻⁴ | 12 | 12 | 25 |
| C3 | 70 | S2 | 1.00.10⁻⁴ | 4.5 | 4.5 | 25 |
| C4 | 73 | S2 | 0.50.10⁻⁴ | 18 | 18 | 40 |
| C5 | 83 | S1 | 3.85.10⁻⁴ | 24 | 0 | 20 |

Copolymers C1 to C5 according to the invention have the characteristics presented in table 2.

TABLE 2
characteristics of copolymers C1 to C5 according to the invention.

| Copolymer | x | y + z | f(pSar) (%) | Mₙ (g/mol) | Ð | H |
|---|---|---|---|---|---|---|
| C1 | 58 | 79 | 19 | 23770 | 1.26 | 1.05 |
| C2 | 63 | 22 | 48 | 9223 | 1.17 | 1.02 |
| C3 | 28 | 9 | 50 | 4961 | 1.22 | 0.96 |
| C4 | 28 | 33 | 21 | 8695 | 1.45 | 1.03 |
| C5 | 66 | 23 | 48 | 9832 | 1.36 | 1.02 |

### Procedure for “One-Pot” Synthesis

Sar NCA (2.78.10⁻² mol, 70 eq) was dissolved in dry DMF at room temperature followed by the addition of neopentylamine as initiator (1 eq). The reaction was stirred at room temperature and after the end of CO₂ evolution, completion of the reaction was confirmed by FTIR spectroscopy.

To the reaction medium containing pSar was added L-GluOBzl NCA (4.52.10-3 mol, 12 eq) and D-GluOBzl NCA (4.52.10-3 mol, 12 eq) previously weighted and mixed. After 20 h at room temperature, total consumption of NCAs was confirmed by FTIR spectroscopy.

Precipitation of the copolymer was performed at room temperature by pouring the reaction mixture on 300 mL of MTBE upon vigorous stirring. After filtration, the product was reslurried with 2×100 mL of MTBE and then dried under vacuum. Copolymer 6 was obtained with a yield of 54%.

The characteristics of copolymer C6 are presented in table 3.

TABLE 3
characteristics of copolymer C6 according to the invention.

| Copolymer | x | y + z | f(pSar) (%) | Mₙ (g/mol) | Ð | H |
|---|---|---|---|---|---|---|
| C6 | 76 | 18 | 58 | 7893 | 1.21 | 1.01 |

### Example 3: Preparation of Copolymers of Formula II

### Procedure for “Reverse One-Pot” Synthesis

L-GluOBzl NCA (11 eq) and D-GluOBzl (11 eq) were dissolved in 40 mL of dry DMF at room temperature followed by the addition of neopentylamine as initiator (58.5 μL, 1 eq). The reaction mixture was stirred at room temperature and after the end of CO₂ evolution, completion of the reaction was confirmed by FTIR spectroscopy.

Sar NCA (70 eq) was then added to the reaction mixture. Again, the reaction mixture has been stirred at room temperature and after the end of CO₂ evolution, completion of the reaction is confirmed by FTIR spectroscopy. Precipitation of the copolymer was performed at room temperature by pouring the reaction mixture on 200 mL of MTBE upon vigorous stirring. After filtration, the product was reslurried with 2×100 mL of MTBE and then dried under vacuum. Copolymer C7 was obtained with a yield of 89%.

Copolymer C8 was prepared following the same procedure as C7 using L-GluOBzl NCA (24 eq) and no D-GluOBzl NCA. Copolymer C8 was obtained with a yield of 91%.

Copolymer C9 was prepared following the same procedure as C7 using D-GluOBzl NCA (24 eq) and no L-GluOBzl NCA. Copolymer C9 was obtained with a yield of 85%.

The characteristics of copolymers C7 to C9 are presented in table 4.

TABLE 4
characteristics of copolymers C7 to C9 according to the invention.

| Copolymer | x | y + z | f(pSar) (%) | Mₙ (g/mol) | Ð | H |
|---|---|---|---|---|---|---|
| C7 | 75 | 25 | 50 | 6985 | 1.38 | 1.02 |
| C8 | 80 | 23 | 53 | 10134 | 1.22 | 1.02 |
| C9 | 70 | 23 | 50 | 9926 | 1.11 | 1.02 |

### Procedure of Capping

Copolymer C8 was dissolved in 10 mL of dry DMF at room temperature followed by the addition of DMAP (16 mg, 1.3.10⁻⁴ mol, 1 eq), and NMM (162 μL, 1.43.10⁻³ mol, 11 eq). After full dissolution, acetic anhydride (123 μL, 1.3.10⁻³ mol, 10 eq) was added and the reaction mixture was stirred overnight at room temperature.

Precipitation of the copolymer C10 was performed at room temperature by pouring the reaction mixture on 50 mL of MTBE upon vigorous stirring. After filtration, the product was reslurried with 2×10 mL of MTBE and then dried under vacuum. Copolymer C10 was obtained with a yield of 83%.

Copolymer C11 was prepared following the same procedure as C10 using C7 as starting material.

Copolymer C11 was obtained with a yield of 87%.

Copolymer C12 was prepared following the same procedure as C10 using C9 as starting material.

Copolymer C12 was obtained with a yield of 81%.

The characteristics of copolymer C10 to C12 are presented in table 5.

TABLE 5
characteristics of copolymer C10 to
C12 according to the invention.

| Copolymer | x | y + z | f(pSar) (%) | Mₙ (g/mol) | Ð | H |
|---|---|---|---|---|---|---|
| C10 | 80 | 23 | 53 | 11442 | 1.19 | 1.02 |
| C11 | 68 | 22 | 50 | 8242 | 1.23 | 1.02 |
| C12 | 68 | 22 | 50 | 10559 | 1.09 | 1.02 |

### Example 4: Preparation of Paclitaxel-Loaded Nanoparticles

Paclitaxel (PTX) loaded nanoparticles were formed by a solvent-displacement method, also called nanoprecipitation.

A solution 1 of copolymer C1 to C12 in DMF at a concentration of 200 g/L was prepared. A solution 2 of PTX in DMF at a concentration of 20 g/L was prepared. Using solutions 1 and 2, a solution 3 at a feed weight ratio (FWR) of 10% was prepared, containing [C]=100 g/L and [PTX]=10 g/L in DMF.

Using a syringe pump (Fusion 100-X, Chemyx), 1 mL of the solution 3 was injected at a flow rate of 30 mL/min in 9 mL of water under 400 rpm stirring. After complete addition, stirring was pursued for 5 min.

The freshly obtained suspensions were purified to remove DMF and unloaded drug by a first filtration on a 0.20 μm PES syringe filter, followed by a gel filtration column (PD-10 desalting, Cytiva) using water as eluent, and a final filtration on a 0.20 μm PES syringe filter. The resulting NP1 to NP12 nanoparticles have the characteristics detailed in table 6.

TABLE 6
characteristics of PTX-loaded nanoparticles
NP1 to NP12 according to the invention.

| | | | | | [PTX in | | DMF |
|---|---|---|---|---|---|---|---|
| Nano- | | Dₕ | | | NPs] | | content |
| particles | Copolymer | (nm) | PDI | LE | (g/L) | LC | (ppm) |
| NP1 | C1 | 83 | 0.26 | 75% | 0.59 | 11% | nd |
| NP2 | C2 | 22 | 0.30 | 67% | 0.52 | 8% | 34 |
| NP3 | C3 | 27 | 0.08 | 68% | 0.51 | 8% | 23 |
| NP4 | C4 | 109 | 0.24 | 68% | 0.43 | 7% | 23 |
| NP5 | C5 | 30 | 0.21 | 84% | 0.58 | 14% | 30 |
| NP6 | C6 | 47 | 0.24 | 64% | 0.46 | / | / |
| NP7 | C7 | 10 | 0.16 | 73% | 0.57 | 8% | 19 |
| NP8 | C8 | 16 | 0.25 | 76% | 0.55 | 12% | 28 |
| NP9 | C9 | 9 | 0.26 | 68% | 0.46 | 8% | 31 |
| NP10 | C10 | 23 | 0.03 | 73% | 0.57 | 11% | 19 |
| NP11 | C11 | 29 | 0.17 | 64% | 0.49 | 7% | nd |
| NP12 | C12 | 23 | 0.03 | 72% | 0.48 | 8% | 17 |

/: not determined
nd: not detected (below limit of detection)

PTX solubility in water is lower than 0.1 μg/mL. Results in table 6 show that nanoparticles according to the invention allow to significantly increase PTX water-solubility.

Advantageously, nanoparticles according to the invention present only traces of DMF, which is compatible with their use in pharmaceutical applications.

1. A linear copolymer comprising a polysarcosine block, pSar, containing from 15 to 99 sarcosine constitutional units and a poly(amino acid) block, pAA, containing from 8 to 120 amino acid constitutional units.

2. The linear copolymer according to claim 1, having a hydrophilic fraction, f(pSar), ranging from 5 to 80%, wherein f(pSar) is the ratio in percentage of number average molar mass of the pSar block on the number average molar mass of the copolymer.

3. The linear copolymer according to claim 1, wherein pAA block has a H hydrophobicity coefficient equal or higher than −0.50.

4. The A-linear copolymer according to claim 1, wherein amino acid constitutional units of pAA block is a hydrophobic amino acid.

5. The A-linear copolymer according to claim 1, of formula I or II: 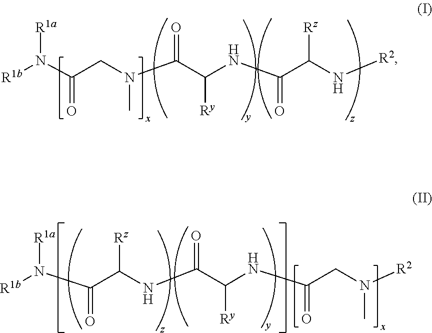
wherein
x is the number of sarcosine constitutional unit and is an integer ranging from 15 to 99,
y+z is the number of amino acid constitutional units and is an integer ranging from 8 to 120,
groups R^{y} and R^{z} are independently chosen from an amino acid lateral-chain group,
groups R^{1a} and R^{1b} are independently chosen from H, alkyl, alkenyl, alkynyl, cycloalkyl, aryl and heteroaryl,
group R² is chosen from H and a nitrogen protecting group.

6. The linear copolymer according to claim 5, wherein x is an integer ranging from 20 to 95.

7. The linear copolymer according to claim 5, wherein y+z is an integer ranging from 8 to 110.

8. The linear copolymer according to claim 5, wherein y+z is an integer ranging from 8 to 50.

9. The linear copolymer according to claim 5, wherein y+z is an integer ranging from 55 to 110.

10. The linear copolymer according to claim 6, wherein R^{y} and R^{z} are independently chosen from the lateral chain of L-leucine, L-phenylalanine, L-tyrosine, γ-benzyl-L-glutamate, γ-tert-butyl-L-glutamate, L-cyclohexylglycine, D-leucine, D-phenylalanine, D-cyclohexylglycine, D-tyrosine, γ-benzyl-D-glutamate, γ-tert-butyl-D-glutamate.

11. A method of preparation of a linear copolymer comprising
polymerizing sarcosine derivates and amino acid derivates, wherein derivates are represented by formulas III and IV as follow 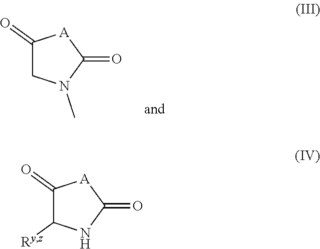
wherein
A is O or S,
R^{y}, R^{z} are as defined in claim 5, and
recovering the linear copolymer.

12. Nanoparticles comprising linear copolymer according to or prepared according to the method of claim 11.

13. Nanoparticles according to claim 12 further comprising at least one active compound.

14. Nanoparticles according to claim 12, satisfying at least one of the following conditions:
a hydrodynamic diameter lower than 400 nm;
a polydispersity index lower than 0.70;
a loading efficiency of the active compound of more than 20%.

15. A method of preparing nanoparticles according to claim 12 comprising the following steps:
preparing an organic solution containing the linear copolymer,
then, mixing the organic solution with an aqueous solution upon stirring,
then, removing the organic solvent.

16. A method of preparing nanoparticles according to claim 15 wherein nanoparticles further comprise an active compound, said method comprising the following steps:
preparing an organic solution containing the linear copolymer and at least an active compound,
then, mixing the organic solution with an aqueous solution upon stirring,
then, removing the organic solvent.

17. The linear copolymer according to claim 2, having a hydrophilic fraction, f(pSar), ranging from 10 and 70%.

18. The linear copolymer according to claim 3, wherein pAA block has a H hydrophobicity coefficient equal or higher than 0.00.

19. The linear copolymer according to claim 4, wherein amino acid constitutional units of pAA block is selected from the group comprising alanine, valine, norleucine, leucine, isoleucine, methionine, phenylalanine, tryptophan, proline, tyrosine, their derivatives, protected hydrophilic amino acids and combinations thereof.

20. Nanoparticles according to claim 13, wherein the at least one active compound is selected from hydrophobic active pharmaceutical ingredients.
